(19) United States
(12) Patent Application Publication
(10) Pub. No.: US 2023/0148737 A1
(43) Pub. Date: May 18, 2023

(54) BEVERAGE DISPENSING DEVICE

(71) Applicant: Merry Hunter, Box Elder, SD (US)

(72) Inventor: Merry Hunter, Box Elder, SD (US)

(21) Appl. No.: 17/529,652

(22) Filed: Nov. 18, 2021

(51) Int. Cl. A45F 3/20

(52) U.S. Cl. CPC A45F 3/20; A61J 1/10

(57) ABSTRACT

A beverage dispensing device, including a main body to store at least one beverage therein, a liquid tube disposed at a first end on at least a portion of the main body to direct the at least one beverage away from the main body, and a dispensing valve disposed on at least a portion of a second end of the liquid tube to dispense the at least one beverage in response to an application of force thereto.

### BACKGROUND

### 1. Field

The present general inventive concept relates generally to hydration, and particularly, to a beverage dispensing device.

### 2. Description of the Related Art

Although some people seek healthcare in healthcare facilities, such as a hospital, some people may suffer severe dehydration due to oversight by medical staff of a patient and/or delays in receiving necessary fluids. Unfortunately, in many hospitals, it is difficult to prioritize service when multiple emergencies occur.

Typically, hospitals and/or nursing homes provide the patient with a large, heavy, and space-consuming cup. However, these large cups are not only inconvenient, but also can be difficult to use, especially for those who are severely injured and/or immobile. As such, for the immobile patient, the cup may be available, but is otherwise inaccessible due to the immobile patient's current physical incapacity.

Therefore, there is a need for a beverage dispensing device that is easily accessible for the patient and does not require extensive physical effort and/or third party assistance to use.

### SUMMARY

The present general inventive concept provides a beverage dispensing device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a beverage dispensing device, including a main body to store at least one beverage therein, a liquid tube disposed at a first end on at least a portion of the main body to direct the at least one beverage away from the main body, and a dispensing valve disposed on at least a portion of a second end of the liquid tube to dispense the at least one beverage in response to an application of force thereto.

The main body may include a slide lock movably disposed on at least a portion of the main body to prevent access to an interior of the main body in response to moving in a first lateral direction, and allow access to the interior of the main body in response to moving in a second lateral direction opposite with respect to the first lateral direction.

The main body may include a thermal compartment disposed on at least a portion of the main body to adjust a temperature level of the main body in response to receiving a thermal pack therein.

The liquid tube may direct the at least one beverage from the main body to the dispensing valve based on a gravitational force in response to the main body being suspended at a higher elevation than the dispensing valve.

The dispensing valve may prevent the at least one beverage from moving therethrough in a first position, and opens in a second position in response to the application of force, such that the at least one beverage moves therethrough.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a perspective view of a beverage dispensing device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the FIGURES, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Beverage Dispensing Device 100
Main Body 110
Slide Lock 111
Thermal Compartment 112
Graduated Markings 113
Hook Aperture 114
Liquid Tube 120
Dispensing Valve 130
Dispensing Aperture 131
Fastener 140

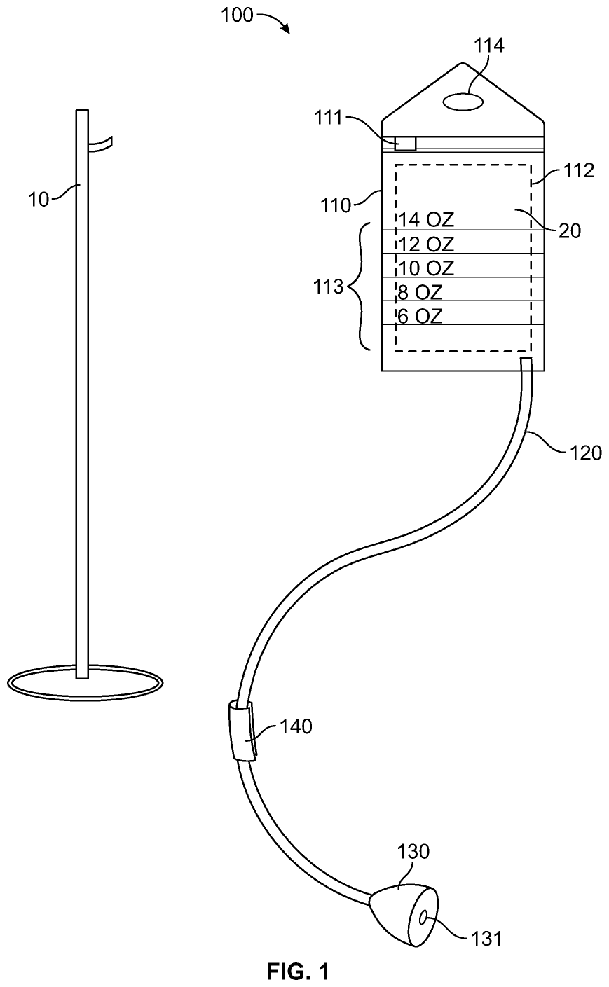

FIG. 1 illustrates a perspective view of a beverage dispensing device 100, according to an exemplary embodiment of the present general inventive concept.

The beverage dispensing device 100 may be constructed from at least one of plastic, metal, wood, glass, and rubber, etc., but is not limited thereto.

The beverage dispensing device 100 may include a main body 110, a liquid tube 120, a dispensing valve 130, and a fastener 140, but is not limited thereto.

The main body 110 may be constructed as a container, such as a bag, a box, a carton, and/or any other type of container, but is not limited thereto. The main body 110 may store at least one beverage therein, such as water, juice, soda, tea, coffee, etc. Also, the main body 110 may be watertight, such that the main body 110 may prevent the at least one beverage from leaking.

The main body 110 may include a slide lock 111, a thermal compartment 112, a plurality of graduated markings 113, and a hook aperture 114, but is not limited thereto.

The slide lock 111 may be movably (i.e., slidably) disposed on at least a portion of the main body 110. The slide lock 111 may prevent (i.e., lock, close) access to an interior of the main body 110 in response to moving in a first lateral direction. Moreover, the slide lock 111 may join and/or close a first side and/or a second side of the main body 110 in response to locking the main body 110. Conversely, the slide lock 111 may allow (i.e., unlock, open) access to the interior of the main body 110 in response to moving in a second lateral direction opposite with respect to the first lateral direction. As such, the first side and/or the second side of the main body 110 may separate from each other in response to unlocking the main body 110.

The thermal compartment 112 may be disposed on at least a portion of the interior of the main body 110. Alternatively, the thermal compartment 112 may be disposed on at least a portion of an exterior of the main body 110 to prevent contact with the at least one beverage. The thermal compartment 112 may also include a fastener (e.g., adhesive, tape, glue, zipper), but is not limited thereto. Thus, the thermal compartment 112 may receive and/or store a thermal pack 20 (e.g., hot pack, cold pack) therein, such that the thermal compartment 112 may be subsequently sealed with the fastener. Additionally, the thermal compartment 112 may have a thermal lining to retain a temperature level based on the thermal pack 20. In other words, the thermal compartment 112 may adjust the temperature level of the main body 110 and/or the at least one beverage in response to receiving the thermal pack 20 therein. As such, the temperature level of the main body 110 and/or the at least one beverage may increase in response to receiving a hot pack. The temperature level of the main body 110 and/or the at least one beverage may decrease in response to receiving a cold pack.

The plurality of graduated markings 113 may be disposed on at least a portion of the main body 110 in a direction parallel to a base of the main body 110. The plurality of graduated markings 113 may identify an amount of the at least one beverage within the main body 110. Furthermore, the plurality of graduated markings 113 may indicate an amount of the at least one beverage that has been consumed.

The hook aperture 114 may be disposed on at least a portion of the main body 110, such that the hook aperture 114 may be disposed on a side of the main body 110 furthest from the base of the main body 110. The hook aperture 114 may receive a hook of a rack 10 therein. Accordingly, the main body 110 may be suspended from the rack 10 while the hook aperture is connected to the rack 10.

The liquid tube 120 may be disposed at a first end on at least a portion of the base of the main body 110. Moreover, the liquid tube 120 may channel the at least one beverage away from the main body 110 through the liquid tube 120.

The dispensing valve 130 may include a dispensing aperture 131, but is not limited thereto.

The dispensing valve 130 may be disposed on at least a portion of a second end of the liquid tube 120. The dispensing valve 130 may be a one-way valve (e.g., leak proof). The dispensing valve 130 may prevent the at least one beverage from moving therethrough in a first position. However, the dispensing valve 130 may open in a second position in response to an application of force thereto (e.g., bite, squeeze, etc.), such that the dispensing aperture 131 may dispense the at least one beverage therefrom. It is important to note that the dispensing valve 130 may create suction within the liquid tube 120 to facilitate extraction of the at least one beverage from the main body 110 in response to the application of force.

In operation, the main body 110 may be suspended from the rack 10. The liquid tube 120 and/or the dispensing valve 130 may be disposed near a user on a bed in a hospital, a nursing home, and/or any other healthcare facility. The dispensing valve 130 may be bitten to dispense the at least one beverage therefrom, such that the user may drink the at least one beverage. Furthermore, a position of the main body 110 being elevated may facilitate extraction of the at least one beverage through the liquid tube 120 to the dispensing valve 130 as a result of a gravitational force. In other words, the liquid tube 120 may direct the at least one beverage from the main body 110 to the dispensing valve 130 based on the gravitational force in response to the main body 110 being suspended at a higher elevation than the dispensing valve 130. Accordingly, the user may avoid using hands and/or moving legs to drink, such as from a cup.

The fastener 140 may include a clamp, a clasp, a cord, a string, a rope, a wire, a bolt, a nut, a washer, an adhesive (e.g., tape, glue), a magnet, and/or any combination thereof, but is not limited thereto.

The fastener 140 may be removably connected to at least a portion of the liquid tube 120. The fastener 140 may removably connect the liquid tube 120 to an external surface, such as a bed rail to facilitate access of the dispensing valve 130 by the user.

Therefore, the beverage dispensing device 100 may facilitate access to the at least one beverage by the user (e.g., a patient). Moreover, the beverage dispensing device 100 may prevent use of hands and/or requiring assistance by a third party to the user.

The present general inventive concept may include a beverage dispensing device 100, including a main body 110 to store at least one beverage therein, a liquid tube 120 disposed at a first end on at least a portion of the main body 110 to direct the at least one beverage away from the main body 110, and a dispensing valve 130 disposed on at least a portion of a second end of the liquid tube 120 to dispense the at least one beverage in response to an application of force thereto.

The main body 110 may include a slide lock 111 movably disposed on at least a portion of the main body 110 to prevent access to an interior of the main body 110 in response to moving in a first lateral direction, and allow access to the interior of the main body 110 in response to moving in a second lateral direction opposite with respect to the first lateral direction.

The main body 110 may include a thermal compartment 112 disposed on at least a portion of the main body 110 to adjust a temperature level of the main body 110 in response to receiving a thermal pack 20 therein.

The liquid tube 120 may direct the at least one beverage from the main body 110 to the dispensing valve 130 based on a gravitational force in response to the main body 110 being suspended at a higher elevation than the dispensing valve 130.

The dispensing valve 130 may prevent the at least one beverage from moving therethrough in a first position, and opens in a second position in response to the application of force, such that the at least one beverage moves therethrough.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A beverage dispensing device, comprising:
a main body to store at least one beverage therein;
a liquid tube disposed at a first end on at least a portion of the main body to direct the at least one beverage away from the main body; and
a dispensing valve disposed on at least a portion of a second end of the liquid tube to dispense the at least one beverage in response to an application of force thereto.

2. The beverage dispensing device of claim 1, wherein the main body comprises:
a slide lock movably disposed on at least a portion of the main body to prevent access to an interior of the main body in response to moving in a first lateral direction, and allow access to the interior of the main body in response to moving in a second lateral direction opposite with respect to the first lateral direction.

3. The beverage dispensing device of claim 1, wherein the main body comprises:
a thermal compartment disposed on at least a portion of the main body to adjust a temperature level of the main body in response to receiving a thermal pack therein.

4. The beverage dispensing device of claim 1, wherein the liquid tube directs the at least one beverage from the main body to the dispensing valve based on a gravitational force in response to the main body being suspended at a higher elevation than the dispensing valve.

5. The beverage dispensing device of claim 1, wherein the dispensing valve prevents the at least one beverage from moving therethrough in a first position, and opens in a second position in response to the application of force, such that the at least one beverage moves therethrough.
